(19) United States
(12) Patent Application Publication
(10) Pub. No.: US 2026/0273157 A1
(43) Pub. Date: Sep. 17, 2026

(54) SYSTEM FOR DRAINAGE

(71) Applicant: SmartCanula LLC, Lausanne (CH)

(72) Inventors: Ludwig Karl Von Segesser, Stansstad (CH); Andreas Terpin, Lausanne (CH)

(21) Appl. No.: 19/469,250

(22) PCT Filed: Mar. 28, 2024
(86) PCT No.: PCT/EP2024/000021

§ 371 Date: Sep. 25, 2025

(30) Foreign Application Priority Data
Mar. 31, 2023 (EP) 23166002.8

(51) Int. Cl. A61M 1/36

(52) U.S. Cl. CPC A61M 1/3659; A61M 2205/3331; A61M 2205/75

(57) ABSTRACT

System for vascular draining comprising a cannula, in particular for use in medical applications, with a distal cannula portion and a proximal cannula portion, wherein the cannula comprises a plurality of flexible filaments, such that the cannula diameter can be varied between a first (D1) and a second diameter (D2), and a tip, a plug and an elongated member, wherein the elongated member comprises the tip at its distal end and the plug at its proximal end to form a mandrel, wherein the plug is configured to be movable positioned at least partially into the proximal cannula portion, and a head element arranged at the distal cannula portion, wherein the head element comprises an opening configured to partially receive the distal end of the mandrel, such that positioning of the plug at the proximal cannula portion varies the cannula diameter between the first (D1) and the second diameter (D2).

### PRIOR ART

Cannulation is essential for extra-corporeal circulation in order to drain blood towards the life support system prior to reinjection into the circulation. For high flow applications like cardio-pulmonary bypass, extra-corporeal membrane oxygenation etc., performance of a cannula can be very important, because it is usually the narrowest part in the perfusion circuit. Conventional cannula designs are typically based on rectilinear designs, i.e., straight tubes. Thus, the resistance of such cannulas is increasing with cannula length in linear fashion. Hence, shorter cannulas can offer better performance. However, with venous cannulas, the tip of the cannula has to be positioned in the right atrium in order to avoid cannula orifice obstruction, thereby creating additional complications.

As a result, two approaches have been developed to improve venous drainage. One approach relates to making the cannula wall thinner in order to get a larger cross-sectional area and thus, providing less resistance. Another approach involves use of augmented venous drainage accomplished through a centrifugal pump or vacuum. However, because the latter approach requires an increased suction it likely results in cannula orifice obstruction and shut off of the venous drainage. This phenomenon is a typical finding in clinical cases undergoing minimal-invasive heart surgery with remote cannulation.

To overcome this problem, the EP 1 248 571 B1 describes a cannula for vascular draining that has a small diameter only for insertion into a vessel. The cannula comprises a cannula body with a lumen extending between the proximal and distal ends of the cannula, wherein the cannula is made of a flexible material, and has at least one mechanism that upon actuation serve to alter the conformation of the cannula between a normal profile conformation in which the cannula has a certain, large lumen diameter for vascular draining, and a low profile conformation in which the lumen diameter of the cannula has been decreased for insertion of the cannula into the vessel of a patient. The cannula therefore comprises a watertight coating at its proximal end, e.g. the flexible material such as a series of interlaced or interwoven wires are coated at the proximal end, whereas at a distal end of the cannula, the blood flow occurs through the uncoated interlaced wires. After proper positioning of the cannula in a vessel, the distal end of the cannula is expanded up to the surface of the interior wall of the vessel for draining. The cannula can be inserted over a guidewire and can be stretched or collapsed using a mandrel, a bougie, a balloon, a pressurization mechanism, or a retraction mechanism. The distal end of the cannula can be stretched with a mandrel.

At the beginning of a drainage procedure, a guide wire is placed in the vessel, to guide the cannula to the desired vessel area. The guide wire is threaded into the cannula through an opening at the distal end of the cannula and then passes inside the cannula. To convert the cannula to a low-profile configuration for insertion into the patient's vessel, i.e., to stretch the flexible wire mesh of the cannula body, a mandrel can be inserted into the lumen of the cannula, i.e. from the proximal end of the cannula up to the distal end of the cannula and over the guidewire. For this purpose, the mandrel may be tubular, to allow the mandrel to be guided over the guide wire for insertion of the mandrel into the lumen of the cannula. At the distal end of the cannula, the mandrel abuts a component. The component forms the tip of the cannula, and the flexible wire mesh is attached to the component. At its proximal end, the mandrel comprises a plug that can be inserted into an opening at the proximal end of the cannula such that, due to the different lengths of the cannula and the mandrel, a force is applied to the component as the mandrel is pressed further into the opening of the cannula. This force tensions the wire mesh and transfers the cannula to the desired low-profile configuration. In the low-profile configuration, the cannula comprises a certain diameter, i.e. the lumen of the cannula has a diameter, which is smaller than in the normal-profile configuration, where the lumen has a larger diameter.

The wire mesh is attached to the component at the distal end of the cannula. To prevent the wire mesh from detaching from the component due to deformation of the component by external forces such as pressure or internal forces such as temperature expansion, the component has mechanical strength, such that deformability due to tensile and compressive forces do not affect the shape of the component. Additionally, because the component forms the distal end of the cannula, it serves to guide the cannula through a vessel and therefore, the component is conically, e.g. tip-shaped, in particular remains tip shaped, when the guide wire is attached to it, to reduce snagging or hooking inside a vessel and further, to reduce the mechanical resistance when the cannula penetrates initially through the small opening in the uppermost skin layers to the vessel. Therefore, because of the various different requirements that are placed on the component based on the application, the component must provide a certain mechanical strength. However, due to the strength of the component and the associated risk of vascular injury, insertion of the cannula through the course of the vessel to the desired position poses a problem.

Furthermore, there is the problem that the guide wire must be pulled out of the cannula through the mandrel before the actual draining begins, and a particularly angled course of the vessel favors kinking of the guide wire. The the opening of the component at the distal end of the cannula through which the guide wire is pulled back has a hard edge, and therefore promotes kinking of the guide wire since the guide wire is first pulled through the tip of the cannula and then through the mandrel.

It is therefore an object of the invention to provide an improved vascular drainage system in which the guidance of the cannula is improved and the risk of vascular injury during insertion of the cannula into the relevant site in the vessel is reduced. It is also intended to reduce the likelihood of kinking of the guide wire, particularly when the wire is being retracted, and/or to facilitate the retraction of a wire that has already kinked. If the guidewire cannot be retracted the entire cannula has to be removed and the procedure restarted.

At the beginning of drainage and as long as the mandrel is still stretching the cannula, i.e. the cannula is in the low-profile configuration, liquid, e.g. blood from the patient, cannot penetrate into the interior of the cannula, since the large openings formed by the wire mesh inside the cannula are blocked by the surface of the mandrel. When the mandrel is removed, and the cannula is transferred to the normal-profile configuration, fluid or blood flows through the openings in the cannula and displaces the air that is partially present there. However, since there must be no air in the drainage circuit, the cannula must have a way to allow the air to escape.

To overcome this problem, the EP 2 341 850 B1 describes that the plug permits passage of air necessary for venting the cannula. Therefore, the plug can be a porous plug, or the plug may be moulded with a slit for venting. The slit has the advantage that it can be easily implemented by a simple modification of the mould for the production of the plug at low cost. However, the slot has the disadvantage that because it is on the outside of the plug, it can easily become contaminated and clogged with impurities.

It is therefore a further object of the invention to provide alternative solutions to the problem of venting the cannula.

### GENERAL DESCRIPTION

The problem is solved by the subject matter of the independent claims, i.e. by a system for vascular drainage according to claim 1, a cannula according to claim 16 and a mandrel according to claim 17 and a kit of parts according to claim 15. Further preferred embodiments of the invention are subject of the dependent claims.

The vascular drainage system according to the invention comprises a cannula, in particular for use in medical applications, with a distal cannula portion and a proximal cannula portion, wherein the cannula comprises a plurality of flexible filaments, such that the cannula can be varied between a normal-profile configuration and a low-profile configuration, and a tip, a plug and an elongated member, wherein the elongated member comprises the tip at its distal end and the plug at its proximal end to form a mandrel, wherein the plug is configured to be movable positioned at least partially into the proximal cannula portion, and a head element arranged at the distal cannula portion, wherein the head element comprises an opening configured to partially receive the distal end of the mandrel, such that positioning of the plug at the proximal cannula portion varies the cannula between the normal and the low-profile configuration.

A drainage is a medical treatment method. It is used to drain or aspirate pathological or increased body fluids or gases in order to restore a normal condition. In principle, a distinction is made between internal and external drainage. In internal drainage, obstacles are surgically, e.g. minimally invasive, bypassed, or accumulated fluids are drained into hollow organs, e.g. the stomach or the intestines. In external drainage, the drainage is from the inside of the body to the outside.

A vascular drainage system, for example according to the “Seldinger-Method” typically comprises a guide wire, inserted into the patient's vessel prior to draining and which serves to guide a cannula to a hollow organ, wherein the cannula is in principle, a flexible hollow tube through which the fluid to be drained is led to the outside of the vessel, i.e. out of the patient.

The cannula is designed to be inserted into a hollow organ, which can be selected from, for example, a vein, an artery, a urethra, a ureter, an intestine, an esophagus, a trachea, a bronchial tube, a pleural space, and/or a peritoneum.

Use of the cannula in medical contexts includes methods such as placing the cannula in its low-profile conformation, inserting the cannula into a hollow organ of a patient at a point of insertion, and returning the cannula to its normal-profile conformation. In the normal profile conformation, the cannula expands, in particular at its distal end, i.e. distal to the point of insertion, up to the diameter of the hollow organ or up to the maximum diameter of the cannula body, i.e. the lumen of the cannula, which is formed by the cannula body.

In the low-profile configuration of the cannula, the diameter of the cannula is reduced for insertion of the cannula into a hollow organ, for example into a vessel compared to the normal-profile configuration. In the low-profile configuration the wire mesh, i.e. the flexible filaments of the cannula form the cannula body, thereby defining a cannula lumen. In the low-profile configuration the flexible filaments are essentially tensioned compared to the normal-profile configuration. The diameter of the cannula refers to the diameter of the lumen, i.e. the inner diameter of the cannula and/or to the diameter of the cannula body, i.e. the outer diameter of the cannula. The diameter relates to the plurality of diameters which appear in the normal-profile configuration by the actual shape of the cannula body and/or lumen, but which alter by tensioning of the cannula when brought into the low-profile configuration.

Therefore, the cannula diameter can be varied between a first diameter (D1) and a second diameter (D2). The first diameter (D1) comprises the low-profile configuration and the second diameter (D2) comprises the normal-profile configuration. When the cannula is in its normal profile conformation when in use, the lumen diameter distal to the point of insertion varies in relation to the diameter of the surrounding vessel. Further, the cannula is in its normal profile conformation when in use, the portion of the cannula distal to the point of insertion supports an inner surface of the surrounding vessel. When the cannula is in its low-profile configuration, a portion of the cannula is characterized by a narrow diameter of the lumen of the cannula that is suitable for insertion into the object to be cannulated as well as into smaller access vessels. However, placing the cannula in the low-profile configuration can be done before, during or after cannulation.

In order to allow for this variation of the cannula diameter, the cannula comprises flexible filaments, i.e. an elastic body formed at least partially from the flexible filaments. The plurality of flexible filaments may include one or more materials selected from metals, shape-memory metals, alloys, plastics, textile fibers, synthetic fibers, and/or combinations thereof. For example, the metal can be stainless steel. Moreover, the plurality of flexible filaments can have a shape selected from round, oval, flattened, triangular, rectangular and combinations thereof. In one embodiment, the plurality of flexible filaments are textile fibers. The plurality of flexible filaments can be braided together, knitted together, or interwoven. Alternatively, the plurality of flexible filaments is interlaced.

The elastic filaments enclose a volume, which can be defined as the inner lumen of the cannula and whose spatial extent can be varied due to the elastic filaments. The lumen has a distal end pointing towards the insertion point and a proximal end, pointing in the opposite direction away from the insertion point, i.e., for example, in a direction away from the patient. Consequently, the cannula comprises a distal cannula portion, which is the portion close to the insertion point and a proximal cannula portion, which is the portion further away from the insertion point.

To change the spatial extent of the lumen of the cannula, i.e. to tension the cannula by conformation from the normal into the low-profile configuration, a mandrel is inserted into the lumen from the proximal cannula portion to the distal cannula portion. To allow the diameter of the cannula, or the lumen of the cannula, to vary between the low-profile configuration and the normal profile configuration, the mandrel engages the distal portion of the cannula and is then pushed further into the lumen of the cannula from the proximal portion, creating a force that tensions the flexible filaments of the cannula and thus changes the diameter of the cannula from the normal-profile configuration to the low-profile configuration. Upon removal of the mandrel, the cannula will expand to its unclamped diameter, in particular by itself due to the tendency of the flexible filaments to assume their original normal shape, like a spring, whereas the normal shape relates to a larger diameter of the lumen at the point of insertion. The mandrel may, in particular for medical applications, comprise a hollow tube and may be configured for insertion of a guide wire, through the hollow tube of the mandrel. In this case, the guide wire passes through the mandrel up to the distal end of the cannula and out through the distal end of the cannula into the hollow organ, e.g. a vein, in order to further guide the cannula to the application site through the vessel, e.g. the vein. The guide wire can then be inserted into and removed from the hollow organ via the mandrel positioned inside the body of the cannula. Further, the mandrel can be removed from the cannula whereas the guide wire remains positioned inside the cannula and the hollow organ.

In order for the mandrel to be suitable for tensioning the cannula, the mandrel includes a tip, a plug and an elongated member. The elongated member may comprise a hollow tube und the tip and the plug of the mandrel may comprise an opening configured for insertion of the guide wire.

The tip is positioned at the distal end of the mandrel. The plug is positioned at the proximal end of the mandrel and the elongated member connects the plug with the tip, thereby forming the mandrel. Therefore, the distal end of the mandrel comprises the tip and the elongated member at least partially, i.e. its distal end. And the proximal end of the mandrel comprises the tip and the elongated member at least partially, i.e. its proximal end. To tension the cannula, the distal end of the mandrel engages the distal end of the cannula. For this purpose, the distal end of the cannula comprises a head element which has an opening. The opening of the head element is designed to partially receive the distal end of the mandrel. This means that a part of the distal end of the mandrel engages in the opening in such a way that a tension can be built up for tensioning the cannula over the mandrel and wherein another part of the distal end of the mandrel does not engage in the opening, for example fits through the opening.

The invention also relates to a mandrel, for use in a cannula with a distal cannula portion and a proximal cannula portion and a plurality of flexible filaments, the mandrel comprises a tip, a plug and an elongated member, wherein the elongated member comprises the tip at its distal end and the plug at its proximal end to form a mandrel, wherein the plug is configured to be movable positioned at least partially into the proximal cannula portion.

The invention also relates to a cannula, in particular for use in medical applications, with a distal cannula portion and a proximal cannula portion, wherein the cannula comprises a plurality of flexible filaments, such that the cannula can be varied between a normal-profile and a low-profile configuration, e.g. between a first (D1) and a second diameter (D2), and a head element arranged at the distal cannula portion, wherein the head element comprises an opening wherein the opening of the head element comprises a first diameter (R1) and a second diameter (R2).

The invention also relates to a kit of parts, comprising a cannula, in particular for use in medical applications, with a distal cannula portion and a proximal cannula portion, wherein the cannula comprises a plurality of flexible filaments, such that the cannula can be varied between a normal-profile and a low-profile configuration, e.g. the cannula diameter between a first (D1) and a second diameter (D2), and a head element arranged at the distal cannula portion, wherein the head element comprises an opening, and a mandrel, comprising a tip, a plug and an elongated member, wherein the elongated member comprises the tip at its distal end and the plug at its proximal end to form the mandrel, wherein the plug is configured to be movable positioned at least partially into the proximal cannula portion, and
wherein the opening of the head element is configured to partially receive the distal end of the mandrel, such that positioning of the plug at the proximal cannula portion varies the cannula between the normal and the low-profile configuration, e.g. the cannula diameter between the first (D1) and the second diameter (D2).

In preferred embodiment of the system, of the mandrel and of the cannula, the tip is configured to partially extend through the opening, such that the tip advantageously supports guiding the cannula through a vessel. Therefore, the head element arranged at the distal end of the cannula comprises an opening large enough to incorporate the tip of the mandrel together with the guide wire. Consequently, when the mandrel is removed, the guide wire has more space in the opening of the head element for moving around. Therefore, if, in a medical application, a guide wire remains in the cannula and the mandrel is removed from the cannula, the guide wire advantageously has a larger entry opening at the distal end of the cannula, which facilitates the extraction of an already kinked guide wire.

In a further preferred embodiment of the system, of the mandrel and of the cannula, the tip is preferably made out of a soft material, e.g. a silicon, which additionally supports guiding of the cannula and due to the softness of the tip reduces the risk of injuring the hollow organ.

In a further preferred embodiment of the system, of the mandrel and of the cannula, the tip comprises a fastening means, in particular a thread, adapted to connect the tip with the distal end of the elongated member. This has the advantage that the tip can be manufactured as a separate component and can therefore be produced cost-effectively in large quantities. Furthermore, different materials of the tip can be quickly and easily exchanged or provided for a particular application.

In preferred embodiment of the system, of the mandrel and of the cannula, the head element is tapered, in particular conically shaped. By making the head element tapered, beveled or conical, the distal end of the cannula is given a streamlined outer contour, making it less likely that the cannula will get caught or snagged during insertion, which reduces the risk of injury when inserting the cannula into the respective hollow organ.

System according to any of the previous claims, wherein the head element comprises a first and a second tapered region. Advantageously, this allows a particularly favorable adaptation of the distal end of the cannula to the tip of the mandrel, so that a substantially smooth transition between the outer surface of the cannula and the surface of the tip can be achieved and the risk of injury when inserting the cannula into the respective hollow organ is thereby reduced.

In this preferred embodiment of the system, of the mandrel and of the cannula, the first tapered region is tapered to adapt to a surface of the distal cannula body portion. I.e., the first tapered region may be shaped to receive the flexible filaments, i.e. the braid of the cannula that partially form the outer skin of the cannula, because, for example, due to the elasticity of the wires, certain shallow angles of curvature allow better attachment of the braid to the first tapered region than steeper ones For better attachment of the wire mesh to the first tapered region, the first tapered region may, for example, be roughened, or comprises a wavy surface, or have certain attachment elements, for example knobs, to incorporate the wire filaments between the knobs.

In this preferred embodiment of the system, of the mandrel and of the cannula, the second tapered region is tapered to adapt to a surface of the tip. I.e. certain steep angles of curvature allow a smoother transition from the second tapered region of the head element onto the surface of the tip of the mandrel. This allows for a particular streamlined shape of the distal end of the cannula, when the mandrel is inserted into the cannula and the cannula is tensioned.

In preferred embodiment of the system, of the mandrel and of the cannula, the opening of the head element comprises a first diameter (R1) configured to receive the tip and a second diameter (R2) configured to clamp the distal end of the elongated member. In this preferred embodiment, the tip may comprise a smaller diameter than the distal end of the elongated member. For example, the tip comprises the diameter R1 and the distal end of the mandrel comprises the diameter R2, for clamping. In this configuration, the tip fits through the opening and the elongated element butts against the opening, so that the mandrel can be tensioned. The tip may comprise a soft material, e.g. a silicon. Alternatively, the tip may comprise a first section which comprises the diameter R1 to fit through the opening and a second section, which comprises the diameter R2, configured not to fit through the opening, i.e. to butt against the opening, so that the mandrel can be tensioned. The tip may comprise a soft material, e.g. a silicon.

In preferred embodiment of the system, of the mandrel and of the cannula the cannula comprises a ventilation opening adapted to compensate for a pressure difference, such that the inside of the proximal cannula portion is in fluid communication with the outside of the cannula. This advantageously allows air, which is trapped within the drainage circuit can escape. A ventilation opening comprises, for example a hole, a channel, or any opening that allows a fluid to flow through for venting.

In preferred embodiment of the system, of the mandrel and of the cannula the ventilation opening comprises a notch and/or a slit arranged in a surface of the plug and/or wherein the ventilation opening comprises a ventilation channel arranged in the plug. In this case, for example, a slit is located on the surface of the plug, wherein the plug comprises a first portion, which is inserted into the proximal end of the cannula body to tighten the cannula and a second portion, which protrudes from the proximal end of the cannula when the cannula is tightened. The slit then extends from the first portion at least partially into the second portion of the plug to allow a fluid to escape from the lumen of the cannula. The slit is located in the surface of the plug which forms a surface contact with the surface of the cannula, i.e. with the proximal end of the cannula, wherein the plug seals the lumen of the cannula at the proximal end of the cannula in a fluid-tight manner.

Alternatively, a channel, for example a through hole is arranged in the plug such that a fluid from inside the cannula lumen can leave the lumen through the channel. The through hole can be configured as a straight hole or as a curved hole, penetrating through the plug. The through hole may additionally comprise a filter material, which for example selectively permits only a certain type of fluid to escape, for example only gaseous fluids escape, wherein any liquids remain inside the lumen. Alternatively, the filter material may act as a semi-permeable membrane, for example only gaseous fluids escape, wherein any liquids remain inside the lumen.

In preferred embodiment of the system, of the mandrel and of the cannula, the ventilation opening comprises the proximal end of the elongated member, wherein the elongated member is tube shaped, to allow ventilation through the elongated member. In this embodiment, the elongated member can perform two tasks simultaneously. On the one hand, the guide wire can be pushed in or out through the cannula by the tube shaped elongated member and on the other hand, a fluid can flow through the hollow-shaped elongated member, in particular when the guide wire is removed.

In preferred embodiment of the system, of the mandrel and of the cannula, the ventilation opening comprises a plurality of apertures arranged in the elongated member. In this context, an aperture comprises an opening, in particular a small opening, which allows, in particular a gaseous, liquid to flow in or out through the aperture. For this purpose, the proximal end of the elongated member comprises, in particular one, or more apertures through which the gas in the lumen can flow out via the elongated member. The cavity of the tube shaped elongated member can merge into an opening of the plug and/or the elongated member closes with the proximal end of the plug, such that the gaseous liquid can flow directly out of the elongated member.

In preferred embodiment of the system, of the mandrel and of the cannula, the plug comprises a filter material, in particular sintered polytetrafluoroethylene, to compensate for a pressure difference, such that the inside of the proximal cannula portion is in fluid communication with the outside of the cannula. The filter material has the property to selectively block liquid fluids and to be transparent for gaseous fluids. The plug can alternatively be completely made from this type of material.

In preferred embodiment of the system, of the mandrel and of the cannula, the plug comprises a first and a second section, wherein the second section is at least partially engaged into the proximal cannula portion and comprises the proximal end of the elongated member and is at least partially conically and/or cylindrically shaped. This embodiment has the advantage that the particularly conical shape of the plug jams the plug at the proximal end of the cannula, so that when the cannula is tensioned by pressing the plug into the proximal end of the cannula like a cork, the plug remains therein at least for a certain period of time and thus the tension of the cannula is automatically maintained over this period of time.

### BRIEF DECRIPTION OF THE FIGURES

FIG. 1 Illustrates a cross-sectional view of the system according to one embodiment, comprising a cannula and a mandrel, wherein the cannula is in a normal configuration and wherein the mandrel is partially inserted into the lumen of the cannula body.

FIGS. 2a, 2b Both figures illustrate the distal end of the mandrel in a cross-sectional view according to one embodiment, comprising a tip and an elongated member, wherein the tip is arranged at the distal end of the elongated member. Additionally, a guide wire is inserted into the mandrel.

FIG. 3 Illustrates a zoom view of the distal end of the system according to one embodiment, comprising a cannula and a mandrel according to one embodiment.

FIG. 4 Illustrates a cross-sectional view of a head element according to one embodiment of the cannula.

FIG. 5 Illustrates a cross-sectional view of the distal end of the system according to one embodiment, comprising a cannula and a mandrel in the low-profile configuration, whereas a guide wire is shown additionally.

FIG. 6 Illustrates an alternative cross-sectional view of the distal end of the system according to one embodiment, comprising a cannula and a mandrel in the low-profile configuration, in particular for venous usage.

FIG. 7 Illustrates a cross-sectional view of the proximal end of the system according to one embodiment, comprising a cannula and a mandrel, wherein the plug comprises a ventilation channel.

FIG. 8 Illustrates a cross-sectional view of the proximal end of the system according to one embodiment, comprising a cannula and a mandrel, wherein the elongated member is hollow shaped, and the plug comprises a plurality of ventilation channels.

FIG. 9 Illustrates a cross-sectional view of the proximal end of the system according to one embodiment, comprising a cannula and a mandrel, wherein the elongated member is hollow shaped, and further comprises a plurality of apertures.

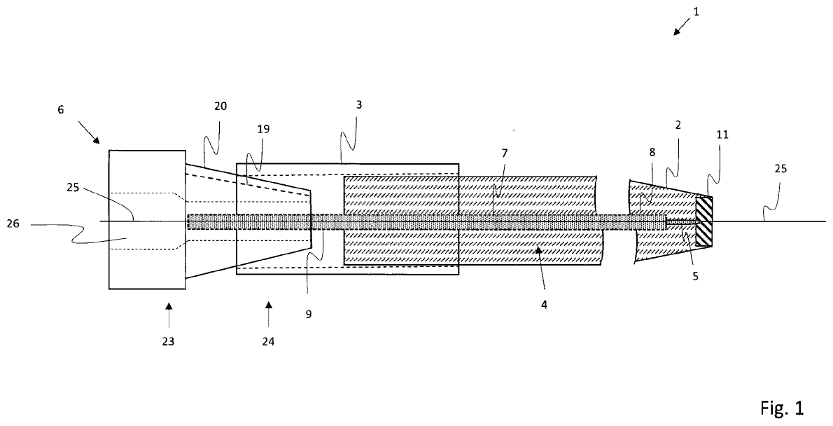

In FIG. 1 a cross-sectional view of the system according to one embodiment is shown, comprising a cannula 1 and a mandrel 10, wherein the cannula 1 is in a normal configuration and wherein the mandrel 10 is partially inserted into the lumen of the cannula body. The cannula body comprises a distal cannula portion 2 and a proximal cannula portion 3, wherein the proximal cannula portion 3 comprises a sleeve 3 to which the plurality of flexible filaments 4, which form the flexible body portion of the cannula 1, are attached. The distal cannula portion 2 comprises a head element 11, which forms the tip of the cannula and to which the flexible filaments 4 are distally attached. The mandrel 10, comprises a plug 6, an elongated member 7, with a distal 8 and a proximal end 9, and a tip 5. The plug 6 connects to the proximal end of the elongated member 9, and the tip 5 connects to the distal end of the elongated member 8. The plug snug fits into the sleeve 3, which forms the proximal cannula portion 3, with its second section 24, thereby sealing the inside of the proximal cannula portion 3 in a fluid tight manner. The first section 23 of the plug remains outside the proximal cannula portion 3. The plug further comprises a notch 19, arranged in the surface of the plug 20. The notch 19 extends from the second section of the plug 24 to the first section of the plug 23, in order to allow for ventilation of a fluid, e.g. a gas, which is inside the cannula 1 and which is pressed outside the cannula 1 through the notch 19, as soon as a further fluid, e.g. blood, is drained through the cannula 1. Therefore, in particular air, which is inside the cannula 1 is released through the notch prior to draining of the blood.

The tip 5 is configured to partially extend through an opening 12 of the head element 11, thereby forming the tip of the cannula at least partially. Therefore, when the mandrel 10 is pulled out of the cannula 1, such that the cannula transfers from the low-profile into the normal-profile configuration, a larger opening 12 remains at the cannula 1 tip compared to the prior art, which advantageously gives the guide wire 25 more freedom of movement and thus facilitates the extraction of the guide wire 25 from the cannula 1. The guide wire 25 is inserted into the plug 6 through the guide wire insertion opening 26. The elongated member 7 of the mandrel 10 is tube shaped to allow the guide wire 25 to extend through the mandrel 10 and the cannula 1, for guiding of the cannula 1 inside a hollow organ, e.g. a vein.

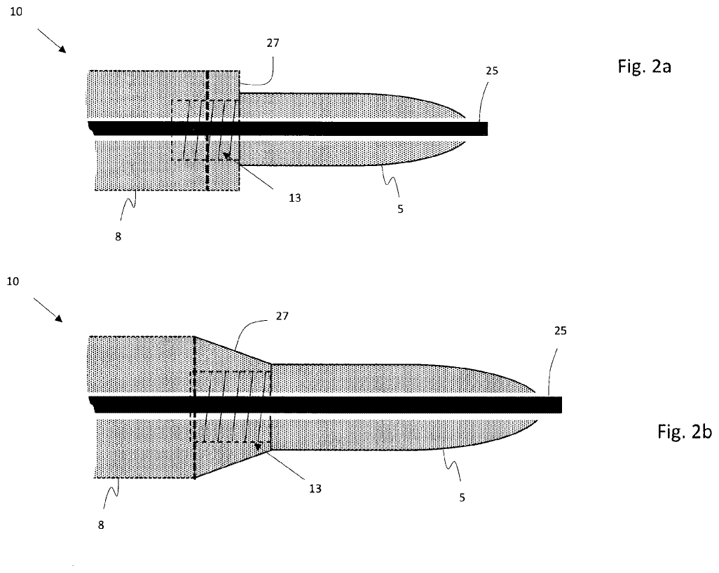

FIGS. 2a, 2b both illustrate the distal end of the mandrel 10 in a cross-sectional view comprising a tip 5 and an elongated member 7 according to one embodiment, wherein the tip 5 is arranged at the distal end of the elongated member 8. Additionally, a guide wire 25 is inserted into the mandrel 10 and extends through the mandrel 10. In FIG. 2a the tip 5 comprises a fastening means 13, e.g. a thread, which serves to connect the tip 5 to the distal end 8 of the elongated member. The tip 5 is, in particular symmetrically, attached to the elongated member 7, preferably the elongated member is formed cylindrically and tube shaped and preferably the tip 5 is shaped cylindrically, whereas the diameter of the tip 5 is smaller than the diameter of the elongated member 7, such that a clamping area 27 remains. The clamping area 27 serves to provide a surface area 27 that abuts a corresponding surface on the head element 11, such that a tension can be established to tension the mandrel 10 inside the cannula 1 in order to transfer the cannula 1 from the normal profile to the low-profile configuration. In an alternative embodiment, the tip 5 comprises the clamping area 27. In this embodiment the tip extends up to the dashed line shown in FIG. 2a, and which indicates, where the distal end of the elongated member 7 and the tip 5 are connected to each other, in particular by use of a fastening means, e.g. a thread 13.

FIG. 2b shows a further alternative embodiment, where the tip 5 is shown attached to conically shaped distal end of the elongated member 8. A guide wire 25 extends through the elongated member 7 and the tip 5. In this embodiment the clamping area 27 is formed by the tapered, i.e. in particular conically shaped region of the elongated member 7. Preferably the tip 5 and/or the elongated member 7 are tube shaped and essentially formed cylindrically and/or at least partially conically. The tip 5 comprises a fastening means 13 to connect to the distal end of the elongated member 8, e.g. a thread. In a further alternative embodiment, the tip comprises the clamping area 27 and therefore the tip extends up to the dashed line shown in FIG. 2b. The thread 13 in this case further extends beyond the clamping area 27 to connect to the distal end of the elongated member 8.

In both FIGS. 2a, 2b the tip 6 may comprise some flexible, soft material, e.g. silicon, which softens the tip and makes it more flexible. Because the cannula tip is at least partially softer and more flexible the risk of injuries during insertion of the cannula 1 into a hollow organ is lower.

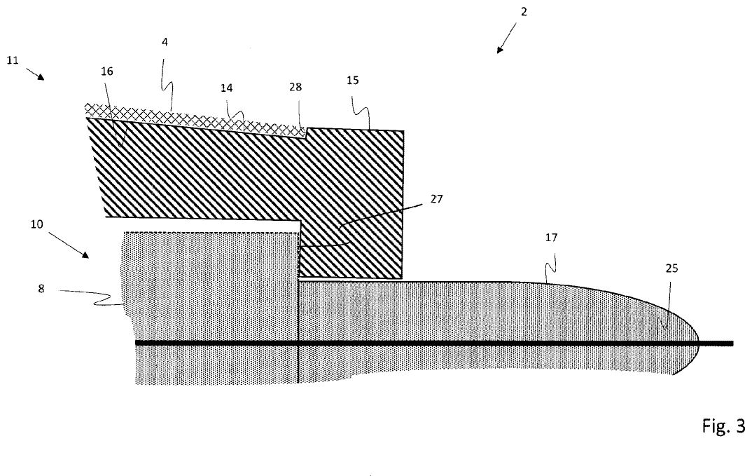

FIG. 3 shows a zoom view according to one embodiment of the distal end of the system, comprising a cannula 1 and a mandrel 10. The figure shows in particular how the clamping area 27 engages into the head element 11 for tensioning of the mandrel 10 inside the cannula 1. A guide wire 25 extends through the mandrel 10. The head element 11 comprises a first tapered region 14 configured to attach to the surface of the distal cannula body portion 16, which is formed by the plurality of flexible elements 4. The plurality of flexible filaments 4 form a wire mesh. For better attachment of the wire mesh to the the tapered region 14 of the head element, the region 14 may be roughened, or comprises a wavy surface, or comprises attachment elements, for example knobs, to incorporate the wire filaments between the knobs. The head element 11 further comprises a second tapered region 15.

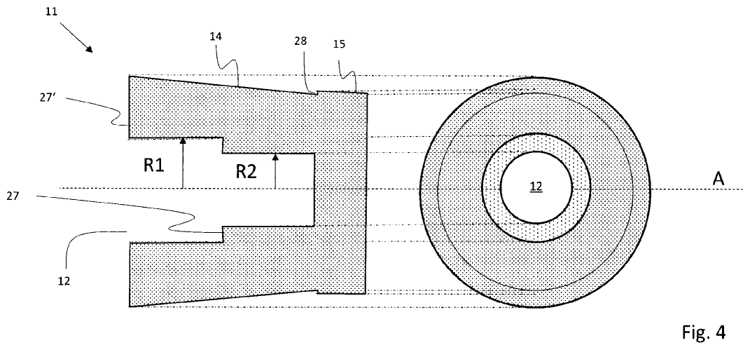

FIG. 4 shows a cross-sectional view of a head element 11 according to one embodiment of the cannula 1. The head element 11 comprises a first opening 12 with a radius R1 and a second opening 12 with a radius R2, wherein in the embodiment as shown in FIG. 4, R1 is larger than R2. Alternative, R1 may be equal to R2 and/or R1 is smaller than R2. In the embodiment as shown in FIG. 4, R1 is larger than R2, which results in the clamping area 27. In the case that R1 equals R2 or in the case where R1 is smaller than R2, the clamping area 27′ results. In the case as shown in FIG. 4, the distal end of the elongated member 8 extends into the opening 12 partially, until the distal end of the elongated member 8 reaches and abuts against the clamping area 27. However, the tip 5, which is attached to the elongated member 7, extends through the opening 12, i.e. the tip 5 extends through the opening sections with radii R1 and R2. Therefore, R2 is configured to be large enough such that the tip 5 extends through the head element 11. In an embodiment, where the tip comprises the clamping area 27, the tip partially extends through the opening 12, such that the clamping area 27 clamps within the opening 12. In this alternative case, the tip 5 partially extends through the section with radius R1 and extends through the section with radius R2. The head element is preferably at least partially formed cylindrically and/or partially formed conically along a symmetry axis A, which is indicated as a dashed line in FIG. 4. The head element 11 comprises a first tapered region 14 and a second tapered region 15, wherein preferably a step 28 appears between the first 14 and second 15 tapered region. The step 28 is configured to be large enough, such that when the flexible filaments 4 are attached to the first tapered region 14 a smooth transition appears between the second tapered region 15 and the surface of the cannula 1, i.e. the surface formed by the plurality of flexible filaments 4, i.e. the wire mesh.

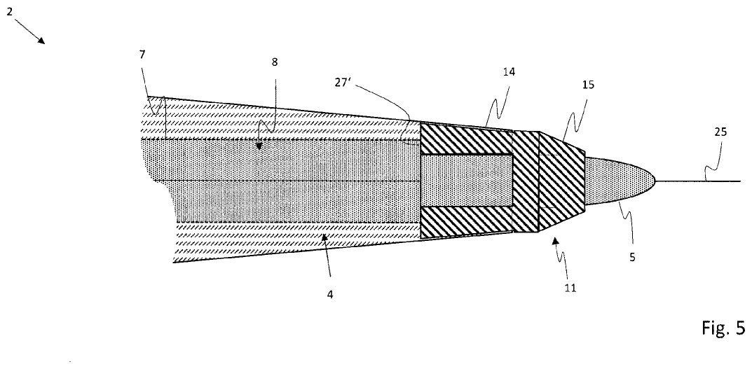

FIG. 5 shows a cross-sectional view of the distal end of the system comprising a cannula 1 and a mandrel 10 in the low-profile configuration, according to one embodiment. The figure focuses of the feature that the head element 11, having the first 14 and second 15 tapered region, is adapted to form a streamlined outer cannula 1 surface, such that guiding of the cannula 1 through a hollow organ along the guide wire 25 is facilitated. Therefore, the first 14 tapered region of the head element 11 is tapered, such that a smooth transition from the flexible filaments 4 to the head element 11 appears. Further, the second 15 tapered region of the head element 11 is adapted to essentially align with the tip 5 of the mandrel, i.e. the part of the tip 5, which extends through the opening 12 of the head element 11, such that the tip of the cannula 1 is formed. In the embodiment shown in FIG. 5, the cannula 1 is in the low-profile configuration. The distal end 8 of the elongated member abuts against the clamping area 27′. In this embodiment, the radii R1 and R2 of the opening 12 of the head element 11 are equal, such that the tip extends through the opening.

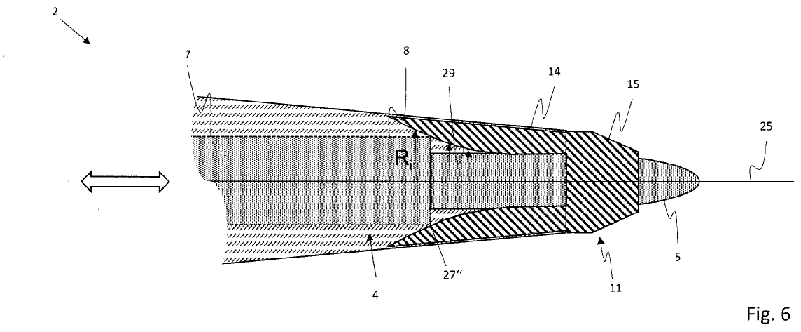

FIG. 6 shows a cross-sectional view of the distal end of the system comprising a cannula 1 and a mandrel 10 in the low-profile configuration, according to one embodiment, in particular for venous use. In this embodiment the fluid, e.g. blood, may flow bidirectional, i.e. into and out of the cannula 1, as it is indicated by the arrow. To reduce or prevent vortex formation and/or inadequate pressure distributions in the outflowing fluid, in particular for medical applications, the head element 11 comprises an extended curved region 29. The extended curved region 29 is adapted to allow for an essentially laminar flow of the fluid within the cannula 1. In this particular embodiment, the opening of the head element comprises a plurality of radii Ri, configured such that the tip 5 extends at least partially through the head element 11 as shown in FIG. 6. The distal end of the elongated member 8 comprises the clamping are 27″. The different types of embodiments of the tip 5 as shown in FIGS. 2a, 2b, are also applicable to the embodiment of FIG. 6, whereas the clamping area 27, when tapered as shown in FIG. 2b, preferably coincides with the curved region, such that an extended clamping surface 27″ results. That is, the clamping surface 27″ extends over several radii Ri of the extended curved region.

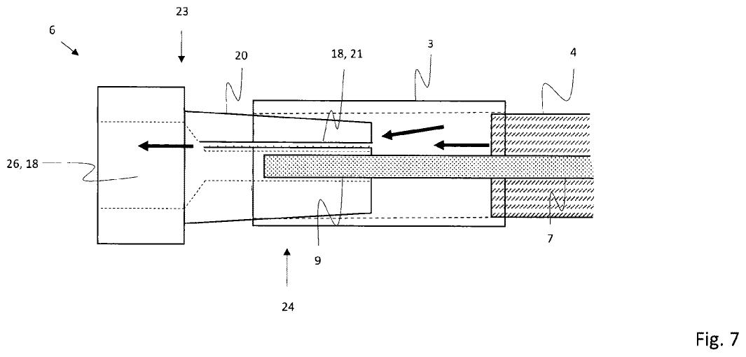

FIG. 7 shows a cross-sectional view of the proximal end of the system according to one embodiment, comprising a cannula 1 and a mandrel 10, wherein the plug 6 comprises a ventilation channel 18, 21. The ventilation channel 21 serves to allow for fluid release, in particular for a gaseous fluid, e.g. air, through the channel 21 prior to draining. The plug 6 is preferably formed cylindrically and/or conically with a first section 23 and a second section 25, whereas the second section 25 snug fits into the proximal cannula portion 3 to seal in a fluid tight manner, the proximal cannula portion 3. The plug further comprises an opening, such that the proximal end 9 of the elongated member can be connected with the plug, and further, a guide wire insertion opening 26, which in the embodiment as shown in FIG. 7, additionally serves as a ventilation opening 18, such that the inside of the cannula 1 is in fluid communication with the outside of the cannula. The ventilation opening 18 therefore comprises the ventilation channel 21 and the guide wire insertion opening 26. The fluid stream, released through the ventilation opening 18, is indicated by arrows in FIG. 7.

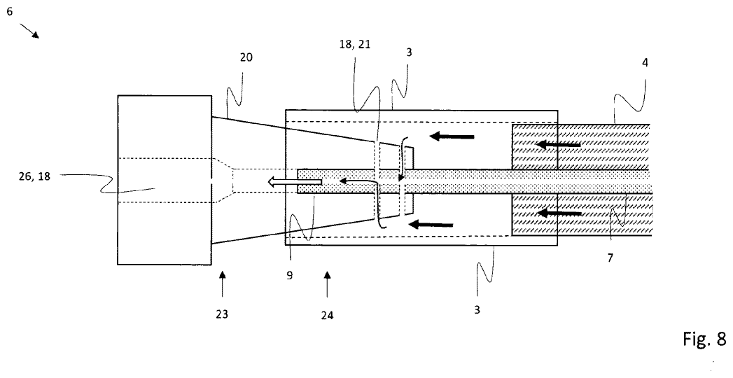

FIG. 8 shows a further embodiment of a ventilation opening 18. In this embodiment, likewise to the embodiment as shown in FIG. 7, the plug comprises a guidewire insertion opening 26, which serves as a ventilation opening, together with a plurality of channels 21, which are arranged in the second section 24 of the plug. As shown in FIG. 8, the second section 24 of the plug is tapered and snug fits into the proximal cannula portion 3. The elongated member 7 is tube shaped to incorporate the guide wire 25 (not shown), but also to allow for a fluid to ventilate trough the elongated member 7. Therefore, the ventilation opening 18 comprise the guide wire insertion opening 26, the channels 21 and at least the distal part 9 of the hollow shaped elongated member. The fluid flow, for releasing a pressure difference between the inside and the outside of the cannula 1, is indicated by arrows in FIG. 8. The channels 21 are shown vertically with respect to the elongated member 7, however any other, e.g. diagonally, arrangement of the channels with respect to the elongated member is possible as long as the inside of the cannula 1 is in fluid communication with the outside of the cannula 1, e.g. means the guide wire insertion opening 26, of the cannula. In FIG. 8, arrows indicate the flow directions for ventilation of the cannula 1.

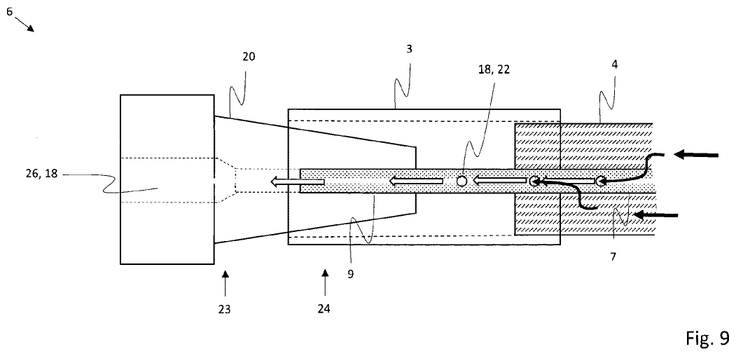

FIG. 9 shows a further embodiment of a ventilation opening 18. In this embodiment, likewise to the embodiments as shown in FIGS. 7, 8 the plug comprises a guidewire insertion opening 26, which serves as a ventilation opening 18 together with a plurality of apertures 22. The apertures 22 are arranged at the proximal end of the elongated member 9, wherein the elongated member 7 is tube shaped, such that a fluid inside the cannula 1 can flow through the apertures 22 into the interior of the at least proximal end of the elongated member 9 to allow for pressure release of the fluid. In the embodiment as shown in FIG. 9, the ventilation opening 18 therefore comprises the proximal end of the tube-shaped elongated member 9, the guide wire insertion opening 26 and the apertures 22, arranged in the proximal end of the elongated member 9. Alternatively, the elongated member 7 can be tube shaped, such that a guide wire 25 can be inserted fully through the elongated member 7. In particular after withdrawal of the guide wire, a fluid can pass through the aperture into the proximal end of the elongated member 9 for ventilation of the fluid. In FIG. 9, arrows indicate the flow directions for ventilation of the cannula 1.

### LIST OF REFERENCE SIGNS

1 cannula
2 distal cannula portion
3 proximal cannula portion
4 flexible filaments
5 tip
6 plug
7 elongated member
8 distal end of the elongated member
9 proximal end of the elongated member
10 mandrel
11 head element
12 opening of the head element
13 fastening means
14 first tapered region of the head element
15 second tapered region of the head element
16 surface of the distal cannula body portion
17 surface of the tip
18 ventilation opening
19 notch
20 surface of the plug
21 ventilation channel
22 apertures
23 first section of the plug
24 second section of the plug
25 guidewire
26 guidewire insertion opening
27, 27′,27″ clamping area
28 step
29 extended curved region of the head element

1. System for vascular draining comprising a cannula (1), in particular for use in medical applications, with a distal cannula portion (2) and a proximal cannula portion (3), wherein the cannula comprises
a plurality of flexible filaments (4), such that the cannula can be varied between a normal profile and a low-profile configuration, and
a tip (5), a plug (6) and an elongated member (7), wherein the elongated member (7) comprises the tip (5) at its distal end (8) and the plug (6) at its proximal end (9) to form a mandrel (10), wherein the plug (6) is configured to be movable positioned at least partially into the proximal cannula portion (3), and
a head element (11) arranged at the distal cannula portion (2), wherein the head element (11) comprises an opening (12), configured to partially receive the distal end (8) of the mandrel, such that positioning of the plug (6) at the proximal cannula portion (3) varies the cannula between the normal and the low-profile configuration.

2. System according to claim 1, wherein the tip (5) is configured to partially extend through the opening (12), such that the tip (5) supports guiding the cannula (1) through a vessel.

3. System according to any of the previous claims, wherein the tip (5) comprises a fastening means (13), in particular a thread, adapted to connect the tip (5) with the distal end of the elongated member (7).

4. System according to any of the previous claims, wherein the head element (11) is tapered, in particular conically shaped.

5. System according to any of the previous claims, wherein the head element (11) comprises a first (14) and a second tapered (15) region, and wherein in particular
the second tapered (15) region is tapered to adapt to a surface (17) of the tip,
or
wherein the head element (11) comprises a first (14) and a second tapered (15) region, wherein the first tapered region (14) is tapered to adapt to a surface (16) of the distal cannula body portion, and wherein in particular the second tapered (15) region is tapered to adapt to a surface (17) of the tip.

6. System according to any of the previous claims, wherein the opening (12) of the head element (11) comprises a first diameter (R1) configured to receive the tip (5) and a second diameter (R2) configured to clamp the distal end of the elongated member (7).

7. System according to any of the previous claims, wherein the cannula (1) comprises a ventilation opening (18) adapted to compensate for a pressure difference, such that the inside of the proximal cannula portion (3) is in fluid communication with the outside of the cannula (1).

8. System according to claim 7, wherein the ventilation opening (18) comprises a notch (19) arranged in a surface (20) of the plug and/or wherein the ventilation opening (18) comprises a ventilation channel (21) arranged in the plug.

9. System according to claim 7 or 8, wherein the ventilation opening (18) comprises the proximal end of the elongated member (9), wherein the elongated member (7) is tube shaped, to allow ventilation through the elongated member (7).

10. System according to claim 9, wherein the ventilation opening (18) comprises a plurality of apertures (22) arranged in the elongated member (7).

11. System according to any of the claims, wherein the plug (6) comprises a filter material, in particular sintered polytetrafluoroethylene, to compensate for a pressure difference, such that the inside of the proximal cannula portion (3) is in fluid communication with the outside of the cannula (1).

12. System according to any of the claims, wherein the plug (6) comprises a first (23) and a second section (24), wherein the second section (24) is at least partially engaged into the proximal cannula portion (3) and comprises the proximal end of the elongated member (9) and is at least partially conically and/or cylindrically shaped.

13. Kit of parts, comprising
a cannula (1), in particular for use in medical applications, with a distal cannula portion (2) and a proximal cannula portion (3), wherein the cannula (1) comprises a plurality of flexible filaments (4), such that the cannula (1) can be varied between a normal profile and a low-profile configuration, and a head element (11) arranged at the distal cannula portion (2), wherein the head element (11) comprises an opening (12),
a mandrel (10), comprising a tip (5), a plug (6) and an elongated member (7), wherein the elongated member (7) comprises the tip (5) at its distal end (8) and the plug (6) at its proximal end (9) to form the mandrel (10), wherein the plug (6) is configured to be movable positioned at least partially into the proximal cannula portion (3), and
wherein the opening (12) of the head element (11) is configured to partially receive the distal end (8) of the mandrel, such that positioning of the plug (6) at the proximal cannula portion (3) varies the cannula between the normal and the low-profile configuration.

14. Cannula, in particular for use in medical applications, with a distal cannula portion (2) and a proximal cannula portion (3), wherein the cannula comprises
a plurality of flexible filaments (4), such that the cannula can be varied between a normal profile and a low-profile configuration, and a head element (11) arranged at the distal cannula portion (2), wherein the head element (11) comprises an opening (12) wherein the opening of the head element comprises a first diameter (R1) and a second diameter (R2).

15. Mandrel, for use in a cannula with a distal cannula portion and a proximal cannula portion and a plurality of flexible filaments (4), the mandrel comprising a tip (5), a plug (6) and an elongated member (7), wherein the elongated member (7) comprises the tip (5) at its distal end (8) and the plug (6) at its proximal end (9) to form a mandrel (10), wherein the plug (6) is configured to be movable positioned at least partially into the proximal cannula portion (3).
